(19) United States
(12) Patent Application Publication
(10) Pub. No.: US 2008/0167600 A1
(43) Pub. Date: Jul. 10, 2008

(54) Device for delivery of an agent to the eye and other sites

(76) Inventor: Gholam A. Peyman, Sun City, AZ (US)

Correspondence Address: THOMPSON HINE L.L.P.;Intellectual Property Group, P.O. BOX 8801, DAYTON, OH, 45401-8801, US

(21) Appl. No.: 11/348,151

(22) Filed: Feb. 6, 2006

Related U.S. Application Data
(63) Continuation-in-part of application No. 11/234,970, filed on Sep. 26, 2005.

(51) Int. Cl. A61M 35/00; A61N 1/30; A61K 39/395; A61P 27/02

(52) U.S. Cl. 604/20; 604/298; 424/142.1

(57) ABSTRACT

A method and device for delivering an agent, such as an anti-vascular endothelial growth factor (VEGF) agent to ameliorate inflammation, at a site in the body that may be the eye, a joint, the brain, etc. The device located at the body site administers agent using an equilibrium diffusion process.

This application is a Continuation-in-Part of U.S. application Ser. No. 11/234,970, filed on Sep. 26, 2005 which is expressly incorporated by reference herein in its entirety.

This application is related to commonly assigned, copending applications, Serial Numbers unknown, each filed Feb. 6, 2006 and entitled DELIVERY OF AN AGENT TO AMELIORATE INFLAMMATION, and DELIVERY OF AN OCULAR AGENT, each naming Peyman as the inventor, each of which is expressly incorporated by reference herein in its entirety.

### BRIEF DESCRIPTION OF THE DRAWINGS

FIG. 1 is a schematic cross-sectional view of a mammalian eye showing a device for delivery of an agent positioned in the eye.

FIG. 2 is a perspective view of one embodiment of a device for delivery of an agent to an eye and other body sites.

FIG. 3 is a cross-sectional view of the device shown in FIG. 2.

FIG. 4 is an enlarged view of the circled area in FIG. 1.

FIG. 5 is a cross sectional view of an alternate embodiment of the device.

FIG. 6 is another alternate embodiment of the device.

FIG. 7 is a cross-sectional view of another alternate embodiment of the device.

FIG. 8 shows one embodiment of the device positioned in the eye.

A method is disclosed for controlling, reducing, or preventing inflammation, an anti-inflammatory response, and/or effects of an anti-inflammatory response, encompassed generally as ameliorating inflammation. The method provides to a patient an anti-vascular endothelial growth factor (VEGF) agent to ameliorate inflammation. Anti-VEGF agents include but are not limited to bevacizumab (rhuMab VEGF, Avastin®, Genentech, South San Francisco Calif.), ranibizumab (rhuFAb V2, Lucentis®, Genentech), pegaptanib (Macugen®, Eyetech Pharmaceuticals, New York N.Y.), sunitinib maleate (Sutent®, Pfizer, Groton Conn.), TNP470, integrin av antagonists, 2-methoxyestradiol, paclitaxel, and P38 mitogen activated protein kinase inhibitors. Anti-VEGF siRNA (short double-stranded RNA to trigger RNA interference and thereby impair VEGF synthesis) may also be used as an anti-VEGF agent.

In one embodiment, the anti-VEGF agent is bevacizumab, administered either alone or with one or more agent(s) known to one skilled in the art under the classification of anti-inflammatory agents. These include, but are not limited to, steroids, anti-prostaglandins, matrix metalloproteinase inhibitors, non-steroidal anti-inflammatory drugs (NSAIDS), macrolides, anti-proliferative agents, anti-cancer agents, etc. In one embodiment, the method ameliorates inflammation using the anti-VEGF agent such as bevacizumab alone. In another embodiment, the method ameliorates inflammation using the anti-VEGF agent such as bevacizumab to supplement known anti-inflammatory agents. In both embodiments, the method ameliorates inflammation at any stage, even early stage inflammation before occurrence of an angiogenic component. The method controls inflammation, and counteracts the action of angiogenic agents such as VEGF on the permeability of a vessel wall, thereby reducing or preventing the resulting tissue damage due to fluid leakage from the vessel (extravasation). The method is applicable to any tissue or site in the body, and to any cause of inflammation such as immune disease including autoimmune disease, viral and/or bacterial infection, trauma including surgical trauma, etc. In one embodiment, the method controls, reduces, or prevents tissue damage in the brain. In one embodiment, the method controls, reduces, or prevents tissue damage in the eye.

Inflammation is a localized, protective response of vascularized tissue to sub-lethal tissue injury or destruction. The response functions to destroy, dilute, or sequester both the injurious agent and the injured tissue. Inflammation can be classified according to duration as either acute or chronic. In the acute form of an inflammatory response, classical signs are pain, heat, redness, swelling, and loss of function. Histologically, there are a complex series of events including dilatation of arterioles, capillaries and venules, with increased permeability and blood flow, exudation of fluids including plasma proteins, and leukocyte migration and accumulation at the site of injury. This reaction may trigger a systemic response such as fever, leukocytosis, protein catabolism, and altered hepatic synthesis of plasma proteins such as C-reactive protein. Chronic inflammation is characterized by macrophage and lymphocyte infiltration into the affected and surrounding tissue.

Inflammation is a homeostatic response to tissue damage by a range of stimuli, including infection and trauma. For example, an inflammatory response helps to destroy or inactivate invading pathogens. In cases of autoimmune diseases such as rheumatoid arthritis, etc., inflammation is a response against self. The inflammatory process removes waste and debris and restores normal function, either through resolution or repair. Tissue structure is normal after resolution, whereas repair leads to a functional, but morphologically altered, organ. In acute inflammation, tissue damage is followed by resolution or healing by scar formation, whereas in chronic inflammation, damage and repair continue concurrently. The initial inflammatory response is usually acute, and may or may not evolve into chronic inflammation. However, chronic inflammation is not always preceded by an acute phase. Although usually beneficial to the organism, inflammation itself may lead to tissue damage, resulting in escalation of chronic inflammation. Inflammation underlies the pathology of virtually all rheumatologic diseases. The severity of disorders, such as arthritis, is classified according to the degree of inflammation and its destructive effects.

Anti-VEGF agents affect the process of angiogenesis, which is the growth of new blood vessels from pre-existing vasculature. It is a fundamental process required for embryogenesis, growth, tissue repair after injury, and the female reproductive cycle. It also contributes to the pathology of conditions such as cancer, age related macular degeneration, psoriasis, diabetic retinopathy, and chronic inflammatory diseases in joints or lungs. Angiogenesis is stimulated when hypoxic diseased, or injured tissues produce and release angiogenic promoters such as VEGF, platelet derived growth factor (PDGF), or fibroblast growth factor (FGF)-1. These angiogenic factors stimulate the migration and proliferation of endothelial cells in existing vessels and, subsequently, the formation of capillary tubes and the recruitment of other cell types to generate and stabilize new blood vessels.

Angiogenic factors may be pro-inflammatory factors. Relatively minor irritation of internal tissues, such as occurs during surgery, does not lead to neovascularization, but encourages tissue adhesion and scarring. Agents that inhibit angiogenesis such as the previously disclosed TNP470, integrin av antagonists, 2-methoxyestradiol, paclitaxel, P38 mitogen activated protein kinase inhibitors, anti-VEGF siRNA, and sunitinib maleate (Sutent®/SU11248) may inhibit synovitis, uveitis, iritis, retinal vasculitis, optic nerve neuritis, papillitis, retinitis proliferance in diabetes, etc. Expression of adhesion molecules such as integrin avb3 and e-selectin are upregulated in new vessels, and new vessels appear sensitive to inflammogens. The angiogenic factor FGF-1 enhances antigen-induced synovitis in rabbits, but is not pro-inflammatory when administered alone. However, angiogenesis occurs in the absence of inflammation such as during embryonic growth and in the female reproductive cycle. Thus, inflammation and angiogenesis can occur independently and administration of anti-VEGF agents such as bevacizumab, either alone or to supplement known anti-inflammatory agents, ameliorates both inflammation without an angiogenic component (earlier stage inflammation), and inflammation that has progressed to an angiogenic component (later stage inflammation). Coexistence of inflammation and angiogenesis may lead to more severe, damaging, and persistent inflammation.

Angiogenesis enhances tumor growth, and anti-angiogenic agents are used clinically. Mechanisms by which new vessels enhance tumor growth include providing metabolic requirements of the tumor, generating growth factors by vascular cells, and inhibiting apoptosis. Inhibiting the function of growth factors such as VEGF can reduce or prevent pathological angiogenesis in tumors.

Angiogenesis may also contribute to thickening of airways in asthma and of lung parenchyma in pulmonary fibrosis, and to growth of sarcoid granulomas. Growth of granulation tissue into airspaces also may be angiogenesis-dependent in bronchi after lung transplant and in alveoli after acute lung injury or in other forms of pulmonary fibrosis. Angiogenesis may also contribute to growth of the synovial pannus in rheumatoid arthritis. Interposition of expanded, innervated synovium between articulating surfaces may contribute to pain on movement. In each of these situations, the expanded tissue may impair function.

The new blood vessels that result from angiogenesis have incomplete walls and are particularly susceptible to disruption and fluid extravasation. This has been proposed as a cause of pulmonary hemorrhage in inflammatory lung disease. Hemosiderin deposits and extravasated erythrocytes are commonly present in inflammatory synovitis, although the contribution of angiogenesis to synovial microhemorrhage is unknown, and its contribution to synovial inflammation remains unclear. The inflammatory potential is evident, however, in patients with hemophilia.

Angiogenesis occurs as an orderly series of events, beginning with production and release of angiogenic growth factors (proteins) that diffuse into nearby tissues. The angiogenic growth factors bind to specific receptors located on the endothelial cells of nearby preexisting blood vessels. Once growth factors bind to their receptors, the endothelial cells are activated and begin to produce enzymes and other molecules that dissolve tiny holes in the sheath-like basement membrane that surrounds existing blood vessels. The endothelial cells begin to divide and proliferate, and they migrate through the holes of the existing vessel towards the diseased tissue or tumor. Specialized adhesion molecules or integrins (avb3, avb5) help to pull the new blood vessels forward. Additional enzymes, termed matrix metalloproteinases (MMP), are produced and dissolve the tissue in front of the sprouting vessel tip in order to accommodate it. As the vessel extends, the tissue is remolded around the vessel. Sprouting endothelial cells roll up to form a blood vessel tube and individual blood vessel tubes connect to form blood vessel loops that can circulate blood. The newly formed blood vessel tubes are stabilized by smooth muscle cells, pericytes, fibroblasts, and glial cells that provide structural support, permitting blood flow to begin.

VEGF is a specific angiogenesis growth factor that binds to receptors on blood vessels and stimulates the formation of new blood vessels. VEGF is a potent inducer of both endothelial cell proliferation and migration, and its biologic activities are largely specific for endothelial and vascular smooth muscle cells. Unlike basic fibroblast growth factor (bFGF), high levels of VEGF are not present in early surgical wounds. Rather, VEGF levels peak seven days after the wound is created, at which point VEGF appears to be a major stimulus for sustained induction of blood vessel growth and high levels of PDGF have been shown. There are abundant sources of VEGF in wounds. Many cell types produce VEGF, including keratinocytes, macrophages, fibroblasts, and endothelial cells. Thus, there is massive VEGF secretion, particularly in the setting of hypoxia, which is often observed in wounds.

Anti-VEGF agents inhibit the action of VEGF. As one example of an anti-VEGF agent, bevacizumab is a recombinant humanized monoclonal IgG1 antibody that binds to and inhibits the biologic activity of human VEGF in in vitro and in vivo assay systems by preventing binding of VEGF with its receptor on the surface of vascular endothelial cells, thus preventing endothelial cell proliferation and new vessel formation. Bevacizumab contains human framework regions and the complementarity-determining regions of a murine antibody that binds to VEGF; it has a molecular weight of about 149 kilodaltons. Bevacizumab, by binding to VEGF, blocks VEGF from binding to receptors and thus blocks angiogenesis. Bevacizumab is typically administered by intravenous infusion, diluted in 0.9% sodium chloride for injection from a 25 mg/ml preparation.

Ranibizumab is a derivative of the full-length antibody bevacizumab (Fab fragment), and is further modified to increase its affinity for VEGF. Both bevacizumab and ranibizumab bind all biologically active isoforms and proteolytic fragments of VEGF, but there are differences. Monovalent binding of a Fab fragment such as ranibizumab to its target antigen would not force the target to dimerize, and hence is useful to manipulate cell receptor function, but its effective antigen binding capacity is lower than its full antibody counterpart. However, VEGF, which is the desired target, is a soluble factor and not a cellular receptor. Therefore, the increased effective binding by the full length antibody bevacizumab enhances inhibition of the VEGF signal and thus provides an enhanced anti-angiogenic effect. Bevacizumab has also been “humanized” to decrease any antigenic effect it may have on the patient, and bevacizumab has a higher molecular weight; this full-length antibody likely will not penetrate the retina to the same extent as the lower molecular weight fragment ranibizumab. However, the increased size of bevacizumab may decrease its clearance rate from the site of action.

Among the available anti-inflammatory agents, many have a target of action to block or ameliorate the actions of pro-inflammatory signals, such as histamine and cytokines. Although this provides some relief from the harmful effects of inflammation, it does not address the cause of the problem. Leukocytes and macrophages, which release pro-inflammatory factors into affected areas, are allowed access to the inflamed tissue following new blood vessel formation.

In one embodiment, the inventive method administers one or a combination of anti-VEGF agent(s) such as bevacizumab, ranibizumab, pegaptanib, etc. as the sole agent(s) to ameliorate inflammation, and thus to control, reduce or prevent an inflammatory response or ameliorate the effects of an inflammatory response. In one embodiment, bevacizumab is used to enhance reabsorption of inflammatory exudates. Decreasing the level of exudates in the eye reduces the inflammatory process and the ensuing hyperpermeable state that occurs with allergies, infection, responses to ocular photodynamic therapy (PDT) and laser treatments, after ocular surgery or trauma, etc. In one embodiment, the anti-VEGF agent is administered to ameliorate an inflammatory process without an angiogenic component. Many inflammatory processes, such as early stage inflammation, are not associated with the formation of new blood vessels. Examples include, but are not limited to, inflammatory diseases of the central nervous system (brain and spinal cord) such as abscess, meningitis, encephalitis, vasculitis, and conditions resulting in cerebral edema; inflammatory diseases of the eye (uveitis, subsequently discussed), macular edema, and others known to one skilled in the art.

In one embodiment, the anti-VEGF agent is administered to ameliorate the scarring and adhesions that are a part of the inflammatory process. Adhesions are bands of scar tissue that bind two internal body surfaces. They are an inflammatory response to tissue damage, and occur as a normal part of any healing process. As one example, adhesions frequently occur during the post-surgical healing process during which tissues have experienced mechanical trauma. However, adverse effects can occur when internal surfaces bind, and adhesions may persist even after the original trauma has healed. Surgery to repair adhesions itself results in recurrent or additional adhesions. The presence of adhesions may also complicate surgical procedures, for example, ocular conjunctival adhesions may complicate subsequent glaucoma surgery.

Adhesions can occur following any type of trauma or surgery, including but not limited to ocular surgery. Examples of ocular surgery that may result in adhesions include glaucoma filtration operations (i.e., iridencleisis and trephination, pressure control valves), extraocular muscle surgery, diathermy or scleral buckling surgery for retinal detachment, and vitreous surgery. Examples of ocular trauma include penetrating ocular injuries, intraocular foreign body, procedures such as PDT, scatter laser threshold coagulation, refractive surgery, and blunt trauma.

In one embodiment, anti-VEGF agents ameliorate disorders with both a vascular proliferative component and a scarring component. As one example, the invention may be used in patients with the ocular disease pterygia. In these patients, fibrovascular proliferation results in scarring of the conjunctiva. An elevated, superficial, external ocular mass, termed a pterygium, forms and extends onto the corneal surface. Patients may experience symptoms of inflammation (e.g., redness, swelling, itching, irritation) and blurred vision. The mass itself may become inflamed, resulting in redness and ocular irritation. Left untreated, pterygia can distort the corneal topography, obscure the optical center of the cornea, and result in altered vision.

The process whereby scar tissue forms (scarring) can occur without new blood vessels being formed (neovascularization). However, the neovascularization process always results in scarring because of the cell proliferation that occurs with the formation of new vessels also results in the proliferation of fibroblasts, glial cells, etc. that result in scar tissue formation. The inventive method may be used to ameliorate the scarring process.

In one embodiment, the anti-VEGF agent is administered to ameliorate inflammation of uveal tissues (uveitis, an inflammation of tissues in the middle layer of the eye, mainly the iris (iritis) and the ciliary body). Ocular inflammation may be associated with underlying systemic disease or autoimmunity, or may occur as a direct result of ocular trauma or infectious agents (bacterial, viral, fungal, etc.). Inflammatory reactions in adjacent tissues, e.g., keratitis, can induce a secondary uveitis. There are both acute and chronic forms of uveitis. The chronic form is frequently associated with many systemic disorders and most likely occurs due to immunopathological mechanisms.

Uveitis presents with ocular pain, photophobia and hyperlacrimation, with decreased visual acuity ranging from mild blur to significant vision loss. Hallmark signs of anterior uveitis are cells and flare in the anterior chamber. If the anterior chamber reaction is significant, small gray to brown endothelial deposits known as keratic precipitates may arise, leading to endothelial cell dysfunction and corneal edema. There may be adhesions to the lens capsule (posterior synechia) or the peripheral cornea (anterior synechia). Granulomatous nodules may appear on the surface of the iris stroma. Intraocular pressure is initially reduced due to secretory hypotony of the ciliary body but, as the reaction persists, inflammatory by-products may accumulate in the trabeculum. If this debris builds significantly, and if the ciliary body resumes its normal secretory output, the pressure may rise sharply, resulting in a secondary uveitic glaucoma.

One skilled in the art will appreciate that scarring and adhesions in areas of the body other than the eye may be treated with the inventive method. Examples include adhesions associated with cardiac surgery (e.g., adhesions in the pericardial space), pulmonary surgery (e.g., in the periplural space), abdominal surgery (e.g., appendectomy, gastric bypass surgery), gynecological surgery (e.g., episiotomy, Caesarean section, hysterectomy), any type of laparoscopy or laparotomy surgery, reconstructive surgery (cosmetic or therapeutic), organ removal (partial or complete), etc.

In another embodiment, the inventive method administers an anti-inflammatory agent simultaneously or concomitantly with an anti-VEGF agent such as bevacizumab and thus controls, reduces, or prevents an inflammatory response. Other anti-VEGF agents such as Lucentis®, Macugen®, Sutent®, geldanamycin, etc. may be included.

The method may be used for any tissue including, but not limited to, eye (e.g., to ameliorate conjunctivitis (inflammation of the conjunctivae, the mucous membranes covering the sclera and inner eyelid), that may be associated with bacterial, viral, or Chlamydia infections, allergies, or susceptibility to irritants such as chemicals, smoke, etc., lung (e.g., to ameliorate interstitial lung disease, inflammation of the interstitium (tissue between the air sacs in the lung)), bone (e.g., to ameliorate synovitis, inflammation of the synovium (the membranes lining joints) that may be associated with arthritis), brain (e.g., to ameliorate encephalitis (inflammation of brain tissue and/or membranes), and muscle (e.g., to ameliorate myopathies (inflammation of muscles, such as muscles near a joint)). The method may be used on patients at risk for developing inflammation. The method may be used on patients with inflammation and/or inflammatory processes from any cause, including but not limited to autoimmune diseases, diseases with an immune component, ischemic diseases, diabetes, age related macular degeneration, retinitis pigmentosa, infectious diseases, allergen-induced inflammation, other degenerative diseases, etc.

In the embodiment where the anti-VEGF agent(s) is administered with an anti-inflammatory agent, an effective amount of the anti-inflammatory agent is administered to a patient at a standard dose known to one skilled in the art. As one example, prednisone is administered for a systemic dose in the range between about 5 mg to about 100 mg daily. As another example, Solu-medrol® is administered intravenously in a single dose of about 1 mg. Other anti-inflammatory agents, possible routes of administration, doses, etc. are known to one skilled in the art. The agent may be administered by any route including enteral and parenteral route, for example, intravenously, orally, ocularly, etc. One skilled in the art will appreciate that the route of administration may vary due to factors such as agent solubility, patient needs, dose required, etc. The anti-inflammatory agent may be a fast-acting anti-inflammatory agent, a slow acting anti-inflammatory agent, or both a fast-acting and a slow-acting anti-inflammatory agent. The anti-inflammatory agent may be formulated for delayed and/or extended release to provide effects over a longer period of time.

Examples of anti-inflammatory agents recognized by one skilled in the art include, but are not limited to, the following: colchicine; a steroid such as triamcinolone (Aristocort®; Kenalog®), anecortave acetate (Alcon), betamethasone (Celestone®), budesonide cortisone, dexamethasone (Decadron-LA®; Decadron® phosphate; Maxidex® and Tobradex® (Alcon)), hydrocortisone methylprednisolone (Depo-Medrol®, Solu-Medrol®), prednisolone (prednisolone acetate, e.g., Pred Forte® (Allergan), Econopred and Econopred Plus® (Alcon), AK-Tate® (Akorn), Pred Mild® (Allergan), prednisone sodium phosphate (Inflamase Mild and Inflamase Forte® (Ciba), Metreton® (Schering), AK-Pred® (Akorn)), fluorometholone (fluorometholone acetate (Flarex® (Alcon), Eflone®), fluorometholone alcohol (FML® and FML-Mild®, (Allergan), FluorOP®), rimexolone (Vexol® (Alcon)), medrysone alcohol (HMS® (Allergan)), lotoprednol etabonate (Lotemax® and Alrex® (Bausch & Lomb), and 11-desoxcortisol; an anti-prostaglandin such as indomethacin; ketorolac tromethamine; ((+)-5-benzoyl-2,3-dihydro-1H-pyrrolizine-1-carboxylic acid, a compound with 2-amino-2-(hydroxymethyl)-1,3-propanediol (1:1) (Acular® Allegan), Ocufen® (flurbiprofen sodium 0.03%), meclofenamate, fluorbiprofen, and the pyrrolo-pyrrole group of non-steroidal anti-inflammatory drugs; a macrolide such as sirolimus (rapamycin), pimocrolous, tacrolimus (FK506), cyclosporine (Arrestase), everolimus 40-O-(2-hydroxymethylenrapamycin), ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, lincosamide, biolimus, ABT-578 (methylrapamycin), and derivatives of rapamycin such as temsirolimus (CCI-779, Wyeth) and AP23573 (Ariad); a non-steroidal anti-inflammatory drug such as derivatives of acetic acid (e.g. diclofenac and ketorolac (Toradol®, Voltaren®, Voltaren-XR®, Cataflam®)), salicylate (e.g., aspirin, Ecotrin®), proprionic acid (e.g., ibuprofen (Advil®, Motrin®, Medipren®, Nuprin®)), acetaminophen (Tylenol®), aniline (e.g., aminophenolacetaminophen, pyrazole (e.g., phenylbutazone), N-arylanthranilic acid (fenamates) (e.g., meclofenamate), indole (e.g., indomethacin (Indocin®, Indocin-SR®)), oxicam (e.g., piroxicam (Feldene®)), pyrrol-pyrrole group (e.g., Acular®), antiplatelet medications, choline magnesium salicylate (Trilisate®), cox-2 inhibitors (meloxicam (Mobic®)), diflunisal (Dolobid®), etodolac (Lodine®), fenoprofen (Nalfon®), flurbiprofen (Ansaid®), ketoprofen (Orudis®, Oruvail®), meclofenamate (Meclomen®), nabumetone (Relafen®), naproxen (Naprosyn®, Naprelan®, Anaprox®, Aleve®), oxaprozin (Daypro®), phenylbutazone (Butazolidine®), salsalate (Disalcid®, Salflex®), tolmetin (Tolectin®), valdecoxib (Bextra®), sulindac (Clinoril®), and flurbiprofin sodium (Ocufen®), an MMP inhibitor such as doxycycline, TIMP-1, TIMP-2, TIMP-3, TIMP-4; MMP1, MMP2, MMP3, Batimastat (BB-94), TAPI-2,10-phenanthroline, and marimastat. The composition may contain anti-PDGF compound(s) such as imatinib mesylate (Gleevec®), sunitinib malate (Sutent®) which has anti-PDGF activity in addition to anti-VEGF activity, and/or anti-leukotriene(s) such as genleuton, montelukast, cinalukast, zafirlukast, pranlukast, zileuton, BAYX1005, LY171883, and MK-571 to account for the involvement of factors besides VEGF in neovascularization. The composition may additionally contain other agents including, but not limited to, transforming growth factor β (TGFβ), interleukin-10 (IL-10), aspirin, a vitamin, and/or an antineoplastic agent.

An effective amount of anti-VEGF agent, either as the sole active agent, or with one or more other non-antiinflammatory agents as previously described, is administered. Administration of either agent may be by any route, and the agents may be administered by the same route or by different routes, including enteral, parental, and ocular routes such as intravitreal injection, subconjunctival injection, retrobulbar injection, topical, etc. As one example, the anti-VEGF agent (bevacizumab, sunitinib, etc.) may be topically administered to intact or compromised eyes, skin, mucous membranes, etc. to reduce scarring after trauma, surgery, radiation, burns, wounds, etc. As another example, it may be locally administered to a site in a surgical field to ameliorate inflammation (e.g., adhesions, scarring, effusions) of pleura, epicardium, etc. after thoracic, cardiac, abdominal, etc. surgery. As another example, it may be administered intrathecally (brain, spinal cord, etc.). As another example, it may be administered by inhalation, for example, to ameliorate inflammation in the respiratory tract (nose, trachea, bronchi, lungs, etc.). As another example, it may be instilled in a body cavity (ventricles, sinuses, bladder, etc.). As another example, sunitinib may be administered systemically (e.g., a single dose/week for one month, then monthly reevaluation of need) or topically (e.g., from about 10 ng/ml to about 100 ng/ml), or intraocularly (e.g., from about 7 ng/ml to about 20 μg/ml). In one embodiment, the administered dose of bevacizumab is less than about 5 mg/0.1 ml. In another embodiment, the administered dose of bevacizumab ranges from 0.1 mg/ml to about 50 mg/ml. In another embodiment, the dose of bevacizumab administered systemically ranges from about 0.05 mg/ml to about 5 mg/ml. In one embodiment, the dose of bevacizumab administered intraocularly (e.g., intravitreally) is about 0.005 mg/0.1 ml to about 5 mg/ 0.1 ml. In one embodiment, the dose of bevacizumab administered topically to the eye is up to 5 mg/ml, and in another embodiment it may be higher. While these doses recite bevacizumab, one skilled in the art will appreciate that they may be used with other anti-VEGF agents, and that doses for a specific agent may be determined empirically, by patient disease severity, other patient variables, etc.

Solutions may be prepared using a physiological saline solution as a vehicle. The pH of an ophthalmic solution may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

The formulations may also contain pharmaceutically acceptable excipients known to one skilled in the art such as preservatives, stabilizers, surfactants, chelating agents, antioxidants such a vitamin C, etc. Preservatives include, but are not limited to, benzalkonium chloride, chlorobutanol, thimerosal, phenylmercuric acetate and phenylmercuric nitrate. A surfactant may be Tween 80. Other vehicles that may be used include, but are not limited to, polyvinyl alcohol, povidone, hydroxypropyl methyl cellulose, poloxamers, carboxymethyl cellulose, hydroxyethyl cellulose, purified water, etc. Tonicity adjustors may be included, for example, sodium chloride, potassium chloride, mannitol, glycerin, etc. Antioxidants include, but are not limited to, sodium metabisulfite, sodium thiosulfate, acetylcysteine, butylated hydroxyanisole, butylated hydroxytoluene, etc. In one embodiment, bevacizumab and/or other anti-VEGF agent(s) may be administered via a controlled release system (i.e., delayed release formulations and/or extended release formulations) such as polylactic or polyglycolic acid, silicone, hema, and/or polycaprolactone microspheres, microcapsules, microparticles, nanospheres, nanocapsules, nanoparticles, etc. A slow release system may release about 10 ng anti-VEGF agent/day to about 50 ng anti-VEGF agent/day for an extended period.

In various embodiments, the compositions may contain other agents. The indications, effective doses, formulations, contraindications, vendors, etc. of these are available or are known to one skilled in the art. It will be appreciated that the agents include pharmaceutically acceptable salts and derivatives.

Administration of an anti-VEGF agent such as bevacizumab, and optionally other agents such as an anti-PDGF agent, another anti-VEGF agent, etc., may supplement or replace PDT and hence avoid the retinal damage frequently associated with PDT. PDT is frequently used to reduce or prevent damage from leaky vessels associated with age related macular degeneration and other diseases. A series of PDT treatments is often performed with a cumulative effect that, over time, results in retinal damage which in some cases may be severe. The present invention may obviate the need for PDT thus eliminating its associated damage.

Bevacizumab at a dose of 5 mg/0.1 ml has been found not to be toxic. In embodiments where bevacizumab or another anti-VEGF agent is administered as the sole agent to ameliorate inflammation, the dose of bevacizumab ranges between about 0.01 mg/0.1 ml to about 5 mg/0.1 ml.

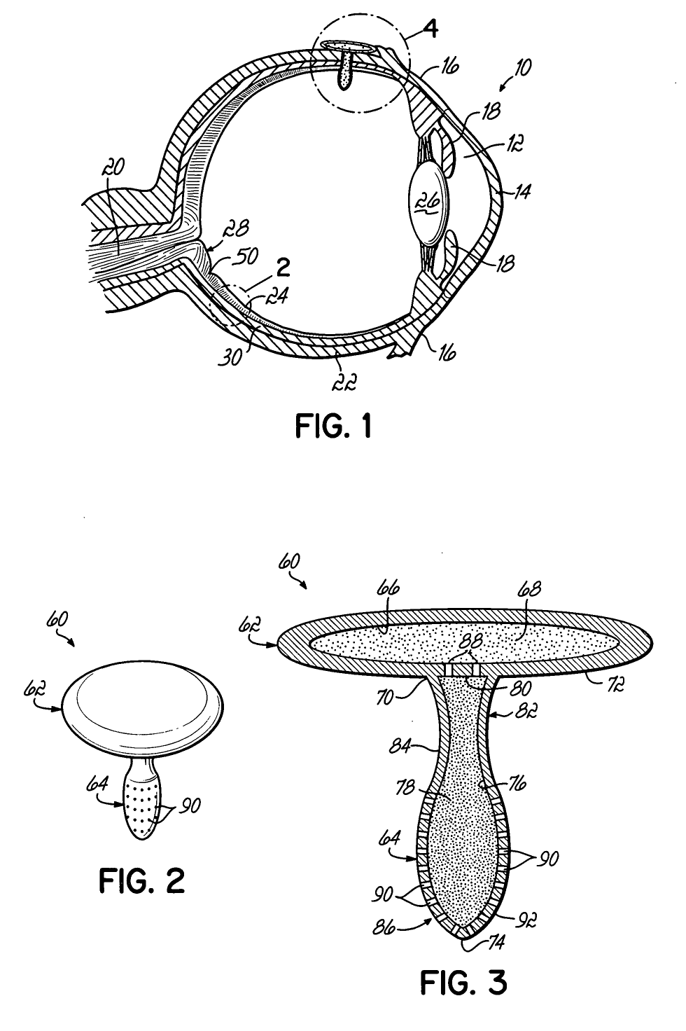

In one embodiment, an agent is administered to the eye via a drug delivery device (FIGS. 1-7). FIG. 1 is a schematic cross-sectional view of a mammalian eye 10 showing the anterior chamber 12, cornea 14, conjunctiva 16, iris 18, optic nerve 20, sclera 22, macula lutea 24, lens 26, retina 28 and choroid 30. In one embodiment, the device may administer a high molecular weight compound, such as bevacizumab, which may be otherwise difficult to administer, dose, etc. While the device is described with reference to delivering bevacizumab, one skilled in the art will appreciate that the device may be used to deliver other agents (e.g., high or low molecular weight agents, non-anti-VEGF agents, etc). In addition, while the use of the device is described with reference to a particular application, e.g., delivery of agent to the eye, one skilled in the art will recognize that the device may be used to deliver agent at other body sites, for example, subcutaneous implantation at or near a joint, spinal cord, subarachnoid space, etc.

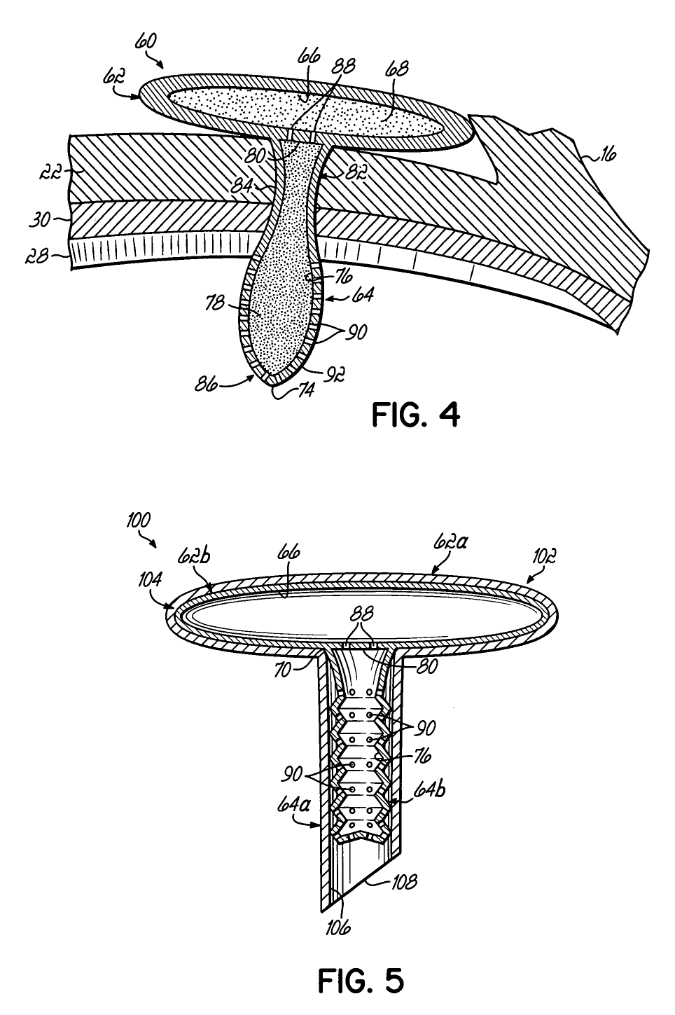

FIG. 2 is a perspective view of an embodiment of the device. FIG. 3 is a cross-sectional view of the device shown in FIG. 1. FIG. 4 is an enlarged view of the circled area 4 in FIG. 1 showing the device positioned in the eye. The device administers agent using an equilibrium diffusion process. The device generally contains a reservoir for the agent to be administered, and is coupled to a fluid filled conduit that, when inserted into the eye, allowing gradual equilibration between the agent and the ocular fluid. Any agent may be administered using the inventive device. In one embodiment, the agent administered using the device has a high molecular weight. In another embodiment, the agent administered using the device is in the form of a suspension. The device in place in the eye may be refilled. For example, a physician may use a needle coupled to a syringe (e.g., 30 gauge needle) to refill the device through the conjunctiva.

More specifically, and as shown in FIGS. 2-4, an exemplary device 60 includes a rigid or semi-rigid proximal portion 62 coupled to a rigid or semi-rigid extension portion 64. The proximal portion 62 may be generally circular, with the diameter including the external portion ranging between about 2 mm to about 7 mm. In one embodiment, the diameter including the external portion is about 3.5 mm. In one embodiment, the diameter including only the internal section ranges between about 0.2 mm to about 5 mm. In one embodiment, the diameter including only the internal section is about 0.5 mm. The invention is not so limited, and one skilled in the art recognizes that the proximal portion 62 may be configured in other shapes having a cross dimension in a similar range. Thus, by way of example and not by limitation, proximal portion 62 may be rectangular, square, octagonal or other suitable shapes. Proximal portion 62 generally defines an interior space or reservoir 66 for containing an agent 68.

Proximal portion 62 is formed from one or more polymers that can be penetrated by a delivery conduit, fine needle, etc. Proximal portion 62 and extension portion 64 may be formed from one or a combination of biocompatible, non-biodegradable polymer materials including, but not limited to, polymethylmethacrylate, a silicone elastomer, silicone rubber, polyolefins such as polypropylene and polyethylene, homopolymers, and copolymers of vinyl acetate such as ethylene vinyl acetate copolymer, polyvinylchlorides, homopolymers and copolymers of acrylates such as polyethylmethacrylate, polyurethanes, polyvinylpyrrolidone, 2-pyrrolidone, polyacrylonitrile butadiene, polycarbonates, polyamides, fluoropolymers such as polytetrafluoroethylene and polyvinyl fluoride, polystyrenes, homopolymers and copolymers of styrene acrylonitrile, cellulose acetate, homopolymers and copolymers of acrylonitrile butadiene styrene, polymethylpentene, polysulfones, polyesters, polyimides, natural rubber, polyisobutylene, polymethylstyrene and other similar biocompatible polymers. One skilled in the art will recognize that proximal portion 62 and extension portion 64 may be formed from the same polymer material or may be formed from different polymer materials, as dictated by the specific application.

In one embodiment, at least one agent 68 in the form of a solution, suspension, etc. is provided in reservoir 66 of proximal portion 62 prior to the device 60 being inserted into the eye 10 (FIG. 1). In another embodiment, agent 68 is provided to reservoir 66 after the device 60 is inserted into the eye 10. The polymer that forms proximal portion 62 may permit resealable penetration by a needle or other conduit to fill the device so that reservoir 66 may be filled/refilled with agent 68 without removing the device 60 from the eye 10.

The extension portion 64 includes a proximal end 70 coupled to a surface 72 of the proximal portion 62 and a distal end 74 opposed to proximal end 70. Extension portion 64 projects away from surface 72 in, for example, a generally perpendicular direction relative to surface 72 and may have any length, typically between about 3 mm to about 6 mm, and a cross dimension, e.g., diameter, typically between about 0.5 mm and about 5 mm. Extension portion 64 includes an interior cavity or space 76 that diffusibly contains a medium (collectively shown schematically at 78 in FIG. 3). For example, medium 78 may be ocular fluid, a combination of ocular fluid and agent 68, or other biocompatible fluids, suspensions, emulsions, etc. There may be open fluid communication between proximal portion 62 with extension portion 64. Alternatively, communication may be regulated, for example, with an adjustable barrier, sieve, or by the physician or patient manually pressing on proximal portion 62 to force agent 68 inside extension portion 64.

An upper portion 82 adjacent proximal end 70 of extension portion 64 has an arcuate shape such that the cross dimension, e.g., diameter, of the extension portion 64 initially decreases but then increases in the distal direction. As shown in FIGS. 3 and 4, this configuration forms an indentation or cavity 84 in extension portion 64 that facilitates securing the device 60 in the eye 10, such as to sclera 22. In this way, the device 60 is not inadvertently withdrawn, dislodged, displaced, loosened, etc. from the eye 10. A lower portion 86 of extension portion 64 terminates at the distal end 74, which may be either sharp or blunt depending on the specific application or physician preference. If the distal end 74 is blunt, the physician may form an incision in the eye 10 to insert the device 60. If the distal end 74 is sharp, the device 60 may be inserted directly into the eye 10 without a separate incision. While the embodiments shown in FIGS. 3 and 4 illustrate a lower portion 86 having a tapered configuration terminating in a sharp or blunt distal end 74, the invention is not so limited, and those of ordinary skill in the art will recognize that other shapes and configurations may be used.

In one embodiment, device 60 operates to administer agent 68 to the eye 10 by equilibrium diffusion. To this end, a diffusible barrier 80 separates the agent 68 in reservoir 66 from the medium 78 in extension portion 64. Diffusible barrier 80 facilitates control of the rate at which agent 68 moves into the medium 78 and is subsequently released into the vitreous cavity, as explained in more detail below. Diffusible barrier 80 includes at least one opening or aperture 88 that permits fluid communication between the reservoir 66 and interior space 76. The aperture(s) 88 may have a wide variety of sizes and configurations depending on the preferences or requirements of a particular application. For example, the aperture(s) 88 may be one or more perforations, fenestrations, holes, slits, and/or slots, and other configurations known in the art. The shape of the aperture(s) 88 may also vary and may be circular, square, rectangular, elliptical, etc. or combinations of shapes. By way of example, FIGS. 3 and 4 show a device 60 where aperture(s) 88 are configured as circular holes.

The size of aperture(s) 88 may be selected depending on the preferences or requirements of a particular application. For example, the aperture(s) 88 may have an identifiable cross dimension (such as diameter, slot length, etc.) that ranges from a few μm up to several mm (e.g., 10 mm). The size of aperture(s) 88 may vary from device to device, and may also vary on the same device. In one embodiment, the device 60 may have walls or other types of closures that selectively reduce or prevent the release of agent 68. The closures may reduce the size of aperture(s) 88 or alternately, completely close aperture(s) 88.

In one embodiment, device 60 may be fabricated to be externally regulated. For example, dosing through the diffusible barrier 80 may be controlled by a software program that communicates with a microchip associated with the device 60. The program may be accessed, verified, altered, monitored, etc., even from a remote location. In embodiments, the release of agent 68 from the device 60 may be pre-set, or may be manually regulated at the point of use, or may regulated from a remote location. This may include volume, duration, rate, release intervals, etc. In one embodiment, the release of agent 68 is remotely controlled by electric stimulation. For example, the aperture(s) 88 may be partially or completely associated with a piezoelectric film, an electric erosion barrier, etc. Upon electric stimulation, the film or barrier is disrupted sufficiently to allow at least a portion of agent 68 in reservoir 66 to egress through the aperture(s) 88. If more than one aperture 88 is present, each aperture 88 may be associated with a film, barrier, etc. that requires different stimulation levels to disrupt, allowing selective control of the delivery of agent 68. The film or barrier may cover all or part of the aperture(s) 88, or be located adjacent an aperture(s) 88, in its association with the device 60. In another embodiment, the release of agent 68 through diffusible barrier 80 is remotely controlled by microactivation, whereby the patient is fitted with a receiving device such as an antenna, and a radiofrequency identification (RF-ID) chip carrying a microactivator for causing the release of agent 68. An RF-ID interrogator is used to interrogate the receiving device, for example, from a remote location, providing power to the RF-ID chip and causing the RF-ID chip to trigger the microactivator by delivering an appropriate coded instruction to the RF-ID chip via radiofrequency signals.

Radio frequency (RF) telemetry may be used to remotely activate the device to release compound 68 through diffusible barrier 80, as known to one skilled in the art. The circuitry, programming, and other components and their implementation are described in, e.g. U.S. Pat. No. 5,170,801 where a circuit in a capsule device receives RF signals and causes drug release from openings in the device; U.S. Pat. No. 5,820,589 where RF telemetry is used to program and/or reprogram power and/or flow rate information to an implanted pump to release a drug, with the pump containing an antenna and circuitry to receive a signal transmitted by an external remote device placed over the skin of the patient; upon receiving a signal, the circuitry changes the operating parameters and the new settings remain in place until new programming instructions are received by RF signals or other non-invasive telemetry in the circuitry; U.S. Pat. No. 5,312,453 describing an external programmer device that transmits RF encoded signals to an implanted device using programming that allows remote selection of parameters and settings for the implanted device; and U.S. Pat. No. 6,824,561, disclosing a hand-held device using RF, infrared, acoustic pulsed, or magnetic activating means where a surgeon, physician, or patient holds the device over the implant site and activates the device to release agent(s). Each of these patents is expressly incorporated by reference herein in its entirety.

These and other embodiments can be adapted by one skilled in the art. As described, the remote activating device may contain a microprocessor and at least one antenna to transmit RF signals to the implanted device. A programming circuit in the implanted device may contain at least one antenna to receive transmitted signals from the remote device and, upon detection of a signal, the programming circuit may cause release of compound 68 from an aperture(s) 88 in the implanted device. As a result, a, physician is able to remotely activate the implanted device to release the compound 68. Additional safety precautions may also be incorporated by one skilled in the art. As one example, the programming circuitry may be configured to respond only to a specific RF signal in order to avoid accidental activation of the implanted device. As another example, the programming circuitry may be configured to incorporate pre-determined dosage information into the remote device in order to prevent remote activation of the implanted device after a maximum dosage has been already released.

RF signals or other telemetry may also serve as a power supply for the implanted device, circuit, and/or any other components. Thus, while operating the remote device, power may be transmitted to the implanted device via the transmitted RF signal, and release of agent 68 may cease when the individual operating the remote device causes it to stop transmitting a signal (i.e., removing the power supply). Various modifications may be made to the embodiments above as known to one skilled in the art.

In one embodiment, the device 60 may be formulated to release agent 68 by electromotive drug administration, also referred to as iontophoresis, using a small electrical current passed through the eye 10. In this embodiment, the inventive device 60 contains an electrode, i.e., an anode and/or cathode depending upon the charge state of the agent 68. An electrode of opposite polarity (cathode and/or anode) is inserted on the sclera 22 at a site opposite device 60. The flow of current is regulated externally to the eye 10 by an energy source. When current is applied, an electrical potential difference is generated between the two electrodes, facilitating movement of agent 68 through diffusible barrier 80 and subsequently released to the eye 10. Such administration may permit a relatively high concentration of agent 68 to be delivered to the affected tissue, rather than being localized at the site of administration. The dose of agent 68 delivered depends upon the current and duration selected. In one embodiment, a current between about 0.5 mA and about 4 mA is applied for between a few seconds to about 20 min.

Iontophoresis itself has no side effects and there is no pain associated with agent administration. Thus, it may be used in any embodiment of the device 60, including those in which the device 60 is externally regulated, and in embodiments where a supratherapeutic concentration of agent 68 is to be delivered.

Once agent 68 diffuses or is otherwise transported into the medium 78 in reservoir 66 of extension portion 64, agent 68 may then diffuse into the vitreous cavity and to the treatment site. To this end, lower portion 86 includes at least one opening or aperture 90 in the outer surface 92 that is in fluid communication with the medium 78 in interior space 76. Similar to the aperture(s) 88, the aperture(s) 90 may have a wide variety of sizes and configurations depending on the preferences or requirements of a particular application. For example, the aperture(s) 90 may be one or more perforations, fenestrations, holes, slits, slots, combinations thereof and other configurations known in the art. The shape of the aperture(s) 90 may also vary. For example, the aperture(s) 90 may be circular, square, rectangular, elliptical, etc. or combinations of these shapes. By way of example, FIGS. 4 and 5 show a device 60 where aperture(s) 90 are configured as circular holes.

The size of aperture(s) 90 may be selected depending on the preferences or requirements of a particular application. For example, the aperture(s) 90 may have an identifiable cross dimension (such as diameter, slot length, etc.) that ranges from a few μm up to several mm (e.g., 10 mm). The size of aperture(s) 90 may not only vary from device to device, but may also vary on the same device. In this way, the smaller opening(s) may be for water only while the larger openings may be for the release of agent 68. As recognized by one skilled in the art, the rate at which agent 68 is released through aperture(s) 90 may be controlled in manner similar to that described above for the release of agent 68 through aperture(s) 88.

In embodiments where an incision is required to place the device 60, the surgery is minimally invasive and is typically performed in a physician's office or on an outpatient basis. An anesthetic is administered to the patient (e.g., topical, local, etc.) as known to one skilled in the art. A relatively small (about 5 mm) incision is made in the peribulbar conjunctiva 16 such that a pocket is created between the conjunctiva 16 and sclera 22. In one embodiment, device 60 is implanted in the pocket such that proximal portion 62 is adjacent sclera 22 and extension portion 64 is positioned in the vitreous cavity. As described, a scleral incision may be used to insert the extension portion 64 within the vitreous cavity. Alternately, the distal end 74 of device 60 may be sufficiently sharp to penetrate the sclera 22 and permit insertion of the device 60 within the vitreous cavity. Although the arcuate shape of extension portion 64 helps secure the device 60 within the eye 10, device 60 may be further secured with the eye 10 by, for example, one or more sutures, using a biocompatible sealant, adhesive, etc. to the scleral wall, or other securing methods.

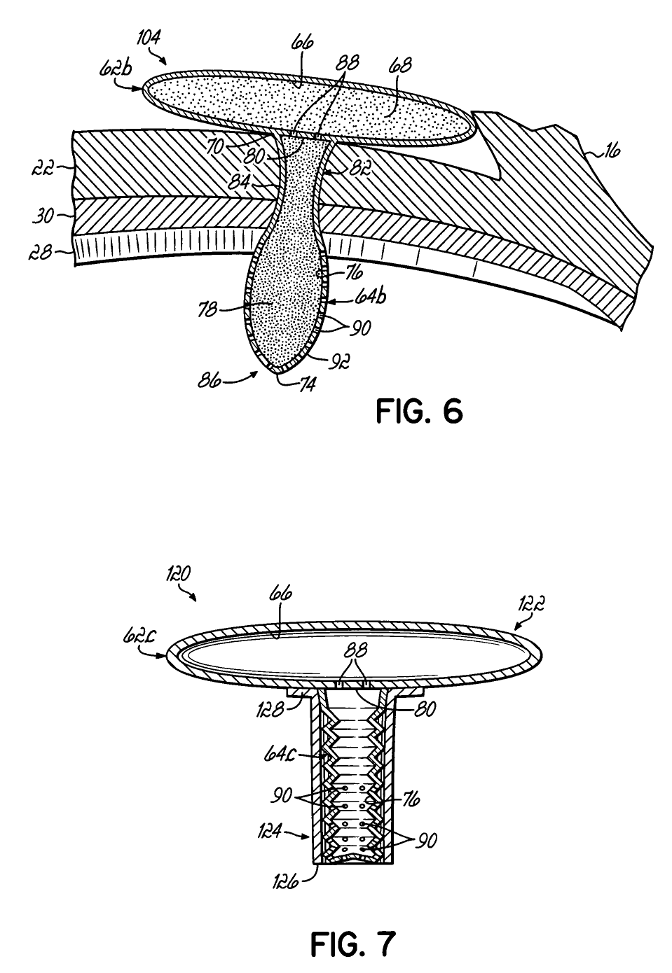

FIGS. 5 and 6, in which like reference numeral refer to like features in FIGS. 2-4, show an alternate embodiment of an intravitreal drug delivery device 100. FIG. 5 is a cross-sectional view of this alternate embodiment. FIG. 6 is a view similar to FIG. 4 showing the device positioned in the eye. Device 100 has an outer relatively rigid shell 102 that encases an inner relatively less rigid bladder 104. For example, the inner bladder 104 may be flexible or semi-flexible. The relative rigidity of the outer shell 102 facilitates surgical insertion of the device 100 through an incision or opening in the wall of the eye, while the less rigid inner bladder 104 reduces irritation of ocular tissue and increases patient comfort. The outer shell 102 may be biodegradable (capable of being degraded in vivo, also termed bioerodible) such that, once inserted, the components of outer structure are substantially absorbed, cleared, etc. by the body, leaving in place the contained inner bladder 104. The inner bladder 104 may be filled or refilled once device 100 is positioned in the eye 10.

More specifically, and as shown in FIGS. 5 and 6, outer shell 102 includes a proximal portion 62a and an extension portion 64a and the inner bladder 104 includes a corresponding proximal portion 62b and an extension portion 64b. With reference to FIG. 5, in one embodiment there is an opening 106 at the distal end 108 of extension portion 64a and, but for this opening, the relatively rigid outer shell 102 substantially encapsulates or surrounds inner bladder 104 and therefore initially gives device 100 a structural aspect. In another embodiment, an opening is not required. As shown in FIG. 5, the distal end 108 of extension portion 64a may be sharp to facilitate insertion of device 100 into the eye, although the invention is not so limited. As described above, outer shell 102 may be formed from a biodegradable polymer. By way of example and not by limitation, outer shell 102 may be formed from biodegradable polymers such as poly(lactic acid), poly(glycolic acid), poly(ortho ester, poly(ethylene-vinyl acetate), polycaprolactone (PCL), a copolymer lactic acid and glycolic acid, a mixture of poly(lactic acid) and poly(glycolic acid), polyanhydrides and their copolymers, and other similar biodegradable polymers.

The inner bladder 104 is less rigid relative to the outer shell 102 and in one embodiment may be flexible or semi-flexible. Proximal portion 62b defines reservoir 66 for holding agent 68. The extension portion 64b may be configured to be expandable between a non-deployed position (FIG. 5), which is capable of being retained within the outer shell 102, and an expanded, deployed state (FIG. 6). For example, as shown in FIG. 5, extension portion 64b may be pleated to give extension portion 64b an accordion-like shape to facilitate expansion of extension portion 64b between the non-deployed and deployed states. Those of ordinary skill in the art will recognize other configurations of extension portion 64b that will permit expansion of inner bladder 104 between the non-deployed and deployed states.

Unlike outer shell 102, inner bladder 104 may be substantially non-biodegradable such that, once inserted, it remains in situ substantially as inserted. The inner bladder may be formed from a biocompatible polymer, such as those provided above, so as to be permeable to diffusion of vitreous fluid and agent. Thus, agent 68 in the reservoir can diffuse from the device into the vitreous cavity, and vitreous fluid can diffuse from the vitreous cavity into the device. Diffusion permits sustained delivery of agent, in contrast to local injection that delivers a bolus of agent, and thus provides certain advantages for the treatment of various ocular conditions.

In operation, device 100 may be implanted into the eye in a manner similar to that described above for the embodiment of FIGS. 2-4. The relatively rigid outer shell 102 facilitates proper placement of the device 100 in the eye with added convenience and without a risk of puncturing, tearing or otherwise destroying the inner bladder 104. Because the outer shell 102 may be biodegradable and the inner bladder is not, the body substantially removes the outer shell 102 leaving the inner bladder 104 positioned in the eye but in a non-deployed state. The reservoir 66 may then be filled with agent 68, such as with an appropriately sized needle inserted into proximal portion 62b via the conjunctiva, and the interior space 76 of extension portion 64b fills with agent 68 and/or vitreous fluid. The agent provided to the device may be a single agent in solution, suspension, etc., or may be a combination of agents in a single solution, suspension, etc., or may be a combination of solutions, suspensions, etc. of different agents, etc. By way of example only, bevacizumab may be administered alone, or in combination with other agents, such as ranibizumab (Lucentis®), pegaptanib (Macugen®), geldanamycin, sunitinib maleate (Sutent®), etc. unbound or bound to microspheres, nanoparticles, etc.

The device 100 then operates in a manner similar to that described above for the embodiment shown in FIGS. 2-4 to deliver agent 68 to a treatment site in the eye 10, that is, by diffusion gradient. Those or ordinary skill in the art will recognize that the various embodiments discussed for regulating the release of agent 68 from the device, such as electronic stimulation, microactivation, RF telemetry and iontophoresis, may also be utilized in the device 100.

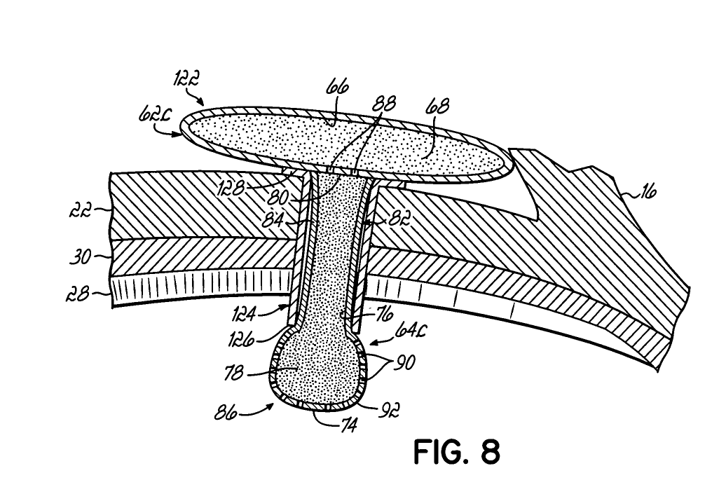

FIGS. 7 and 8, in which like reference numerals refer to like features in FIGS. 2-6, show another embodiment of an intravitreal drug delivery device 120 similar to that shown in FIGS. 5 and 6. FIG. 7 is a cross-sectional view of this alternate embodiment. FIG. 8 is a view similar to FIG. 4 showing the device positioned in the eye. Device 120 includes a flexible or semi-flexible bladder 122 substantially similar to inner bladder 104 and an outer relatively more rigid sleeve 124 that encases a portion of the bladder 122. The relative rigidity of sleeve 124 facilitates surgical insertion of the device 120 through an incision or opening in the wall of the eye and promotes greater stability of the device 120 once positioned in the eye. Unlike the previous embodiment, however, the outer sleeve 124 is substantially non-biodegradable.

The bladder 122 is less rigid relative to sleeve 124 and includes proximal portion 62c defining reservoir 66 for holding agent 68. As with the previous embodiment, the extension portion 64c is configured to be expandable between a non-deployed position (FIG. 7) that is capable of being retained within sleeve 124 and an expanded, deployed state (FIG. 8) wherein lower portion 86 extends beyond an end 126 of sleeve 124. For example, as shown in FIG. 7, extension portion 64c may be pleated to give extension portion 64c an accordion-like shape to facilitate expansion of extension portion 64c between the non-deployed and deployed states. Those of ordinary skill in the art will recognize other configurations of extension portion 64c that will permit expansion between the non-deployed and deployed states.

As shown in FIGS. 7 and 8, the sleeve 124 substantially encapsulates or surrounds an upper portion 82 of extension portion 64c and provides device 120 with a structural aspect. Sleeve 124 may be formed from a biocompatible polymer, such as those provided above, so as to have sufficient rigidity. The bladder 122 may likewise be formed from a biocompatible polymer, such as those provided above, so as to be permeable to diffusion of vitreous fluid and agent. Thus, agent in the reservoir can diffuse from the device into the vitreous cavity, and vitreous fluid can diffuse from the vitreous cavity into the device. A proximal end 128 of sleeve 124 is coupled to proximate portion 62c of bladder 122 and contains extension portion 64c when bladder 122 is in a non-deployed state.

In use, device 120 may be implanted into the eye in a manner similar to that described above for the embodiment of FIGS. 2-4. The relatively rigid sleeve 124 facilitates proper placement of the device 120 in the eye with added convenience. A device with the reservoir prefilled with agent may be positioned in the eye. Alternatively, once positioned in the eye, the reservoir 66 is then provided with agent 68, such as with an appropriately sized needle or other conduit inserted into proximate portion 62c via the conjunctiva, and the interior space 76 of extension portion 64c fills with agent 68 and/or vitreous fluid by diffusion. As the extension portion 64c fills with agent and/or vitreous fluid, the extension portion 64 expands to its deployed state wherein the lower portion 86 extends beyond the distal end 126 of sleeve 124 and within the vitreous cavity. The device 120 then operates in a manner similar to that described above for the embodiments shown in FIGS. 2-6 to deliver agent 68 to the eye 10. Those of ordinary skill in the art will recognize that the various embodiments discussed for regulating the release of agent 68 from the device, such as electronic stimulation, microactivation, RF telemetry and iontophoresis, may also be utilized in the device 120.

It should be understood that the embodiments of the present invention shown and described in the specification are only preferred embodiments of the inventor who is skilled in the art and are not limiting in any way. As one example, the inventive method may be used to treat cerebral edema associated with meningitis by intravenously administering bevacizumab or another anti-VEGF agent. As another example, the inventive device may be used to administer a high molecular weight agent or low molecular weight agent in suspension to a joint. Therefore, various changes, modifications or alterations to these embodiments may be made or resorted to without departing from the spirit of the invention and the scope of the following claims.

What is claimed is:

1. A device for delivering an agent, the device adapted to have at least a portion positioned within a fluid-filled body part, the device comprising
a proximal portion having a reservoir adapted to contain an agent;
an extension portion coupled to the proximal portion and adapted to be positioned within the body part containing fluid, the extension portion including an interior space for containing the agent and at least one aperture in an outer surface of the extension portion and in fluid communication with the interior space; and
an adjustable barrier juxtaposed between the reservoir of the proximal portion and the interior space of the extension portion, the barrier having at least one aperture for permitting fluid communication between the reservoir and the interior space, wherein the aperture is adapted to control the rate at which the agent enters the extension portion and thereafter equilibrates with the fluid in the body part.

2. The device of claim 1 wherein the proximal and extension portions of the device further comprise a relatively more rigid outer shell formed from a substantially biodegradable polymer for providing a structural aspect to the device; and
a relatively less rigid inner bladder formed from a substantially non-biodegradable polymer for permitting diffusion of the agent into the fluid, the outer shell initially substantially encapsulating the inner bladder.

3. The device of claim 1 wherein the proximal and extensions portions of the device further comprise
a bladder formed from a substantially non-biodegradable polymer for permitting diffusion of the agent, the bladder expandable between a first position and a second position; and
a sleeve formed from a substantially non-biodegradable polymer for providing a structural aspect to the device, the sleeve substantially encapsulating the extension portion when the bladder is in the first position and at least a portion of the bladder extending beyond an end of the sleeve when in the second position.

4. The device of claim 1 further comprising an electrode associated with the device for iontophoresis administration of the agent.

5. An ocular device for delivering an agent the device comprising
a proximal portion having a reservoir adapted to contain an agent;
an extension portion coupled to the proximal portion and adapted to be positioned within a vitreous cavity containing vitreous fluid, the extension portion including an interior space for containing the agent and at least one aperture in an outer surface of the extension portion and in fluid communication with the interior space; and
an adjustable barrier juxtaposed between the reservoir of the proximal portion and the interior space of the extension portion, the barrier having at least one aperture for permitting fluid communication between the reservoir and the interior space, wherein the aperture is adapted to control the rate at which the agent enters the extension portion and thereafter equilibrates with vitreous fluid in the vitreous cavity.

6. The ocular device of claim 5 wherein the proximal and extension portions of the device further comprise
a relatively more rigid outer shell formed from a substantially biodegradable polymer for providing a structural aspect to the device; and
a relatively less rigid inner bladder formed from a substantially non-biodegradable polymer for permitting diffusion of the agent into the fluid, the outer shell initially substantially encapsulating the inner bladder.

7. The ocular device of claim 5 wherein the proximal and extensions portions of the device further comprise
a bladder formed from a substantially non-biodegradable polymer for permitting diffusion of the agent, the bladder expandable between a first position and a second position; and
a sleeve formed from a substantially non-biodegradable polymer for providing a structural aspect to the device, the sleeve substantially encapsulating the extension portion when the bladder is in the first position and at least a portion of the bladder extending beyond an end of the sleeve when in the second position.

8. The ocular device of claim 5 further comprising an electrode associated with the device for iontophoresis administration of the agent.

9. A method of ameliorating an ocular inflammatory process in a patient comprising providing to an eye of the patient a therapeutic concentration of at least one anti-vascular endothelial growth factor (VEGF) agent as the anti-inflammatory agent, the agent provided with a device to achieve a diffusion concentration of the anti-VEGF agent in the vitreous cavity at a concentration sufficient to ameliorate ocular inflammation.

10. The method of claim 9 wherein that anti-VEGF agent is bevacizumab administered at a concentration of up to about 5 mg/0.1 ml.

11. The method of claim 9 wherein the anti-VEGF agent is selected from at least one of bevacizumab, ranibizumab, pegaptanib, sunitinib maleate, TNP470, integrin av antagonists, 2-methoxyestradiol, paclitaxel, or P38 mitogen activated protein kinase inhibitors.
